(19) United States
(12) Patent Application Publication
(10) Pub. No.: US 2022/0370701 A1
(43) Pub. Date: Nov. 24, 2022

(54) BIDIRECTIONAL PERFUSION CANNULA

(71) Applicant: MEDTRONIC, INC., Minneapolis, MN (US)

(72) Inventors: Victor Duijsens, Grevenbicht (NL); Paul Venrooij, Hoensbroek (NL); Patrick Kleijnen, Mheer (NL); Luc Alleleyn, Wittem (NL); Paul Adams, Munstergeleen (NL); Sevada Avaksoumbatian, Bussigny (CH)

(73) Assignee: MEDTRONIC, INC., Minneapolis, MN (US)

(21) Appl. No.: 17/770,488

(22) PCT Filed: Sep. 17, 2020
(86) PCT No.: PCT/EP2020/076010

§ 371 Date: Apr. 20, 2022

(30) Foreign Application Priority Data
Oct. 22, 2019 (EP) 19000481.2
Oct. 22, 2019 (EP) EP19000482.0

(51) Int. Cl. A61M 1/36; A61M 25/00

(52) U.S. Cl. CPC A61M 1/3659; A61M 25/007; A61M 1/3666; A61M 25/0032; A61M 2025/0037; A61M 2025/0031; A61M 2025/0166

(57) ABSTRACT

The present invention provides a bi-directional perfusion cannula for use in peripheral veno-arterial extracorporeal membrane oxygenation of a patient, the cannula comprising a cannula main body having a primary lumen, the primary lumen leading to a distal end of the cannula main body for providing retrograde blood perfusion, characterized in that the main body further comprises a passage for passing a second cannula therethrough, the passage being oriented such that when a second cannula is inserted into the passage the second cannula is arranged for providing anterograde blood perfusion.

The present invention relates to a system suitable for use in a veno-arterial extracorporeal membrane oxygenation (VA-ECMO) treatment of a patient.

Peripheral VA-ECMO is used worldwide as a temporary cardio-respiratory support in emergency situations. During VA-ECMO venous blood is drained from the venous circulation and it is returned to the arterial circulation, through cannulas. In VA-ECMO, retrograde blood flow (i.e. blood flow towards the upper body) is achieved, while anterograde flow (i.e. blood flow towards the lower limbs) is generally not achieved.

As a result, lower limb ischemia occurs, being a frequent (10%-60%) and dangerous complication, increasing morbidity and mortality. To eliminate this challenge in clinical practice, pediatric distal leg perfusion cannulas are used to ensure concomitant perfusion of the limb, which is however not an optimal solution.

A variety of ECMO techniques have been implemented to date. These techniques include veno-venous (VV) ECMO and veno-arterial ECMO. In VV-ECMO, blood is drawn from the venous system, oxygenated and then returned to the venous system. Typically the blood is drawn from the femoral vein and reintroduced into the jugular vein. In VA-ECMO, blood is typically drawn from the femoral vein and reintroduced into the femoral artery in retrograde fashion. In VA-ECMO, a third catheter may be used. Peripheral VA-ECMO is distinguished from Central VA-ECMO in which direct atrial cannulation is performed.

Such techniques are summarized in Ann Transl Med 2017 Feb.; 5(4) 70 available at https://www.ncbi.nlm.nih.gov/pmc/articles/PMC5337209/.

Peripheral cannulas for ECMO are also reviewed by Svitlana Strunina et al. in “The peripheral cannulas in extracorporeal life support”, Biomed. Eng./Biomed. Tech 2019; 64(2): 127-133, available at https://doi.org/10.1515/bmt-2017-0107.

Dual lumen cannulas for ECMO treatment are known. Reference is made to arrangements described, for example, in WO 2018/091474 A1. US 2018/0043085 A1 describes a single lumen cannula with two outlet ports, a main distal outlet and a side outlet.

A further arrangement for a bi-directional cannula is described in a paper by Yi Chen et al. entitled “Pressure and Flow Characteristics of a Novel Bidirectional Cannula for Cardiopulmonary Bypass”, Innovations 2017; 12:430-433. This paper describes a cannula having an angled shoulder with a side hole for distal limb perfusion.

The present invention provides a bi-directional perfusion cannula for use in peripheral veno-arterial extracorporeal membrane oxygenation of a patient, the cannula comprising a cannula main body having a primary lumen, the primary lumen leading to a distal end of the cannula main body for providing retrograde blood perfusion, characterized in that the main body further comprises a passage for passing a second cannula therethrough, the passage being oriented such that when a second cannula is inserted into the passage the second cannula is arranged for providing anterograde blood perfusion.

The passage may be a conduit internal to the main body or within a sidewall of the main body. Alternatively, the passage may be provided by holes on either side of the main body such that the second cannula may cross the main body.

Seals are provided to prevent the leakage of blood caused by the passage, the seals being arranged to seal around the inserted second cannula.

The invention also provides a bi-directional perfusion cannula for use in peripheral veno-arterial extracorporeal membrane oxygenation of a patient, the cannula comprising a cannula main body having a superior lumen and an inferior lumen, wherein the superior lumen extends to a distal end of the cannula for providing retrograde blood perfusion, characterized in that the cannula further comprises a flexible tube extending from the cannula main body in a direction away from the distal end, the flexible tube being in fluid communication with the inferior lumen for providing anterograde blood perfusion.

In a still further aspect, the invention provides a bi-directional perfusion sleeve having a first lumen for receiving a retrograde perfusion catheter to form a reperfusion cannula and a second lumen for receiving an anterograde perfusion catheter to form a bi-directional cannula, the sleeve having at a first end a first opening to the first lumen and a first opening to the second lumen and at a second end a second opening to the first lumen and between the first end and the second end a second opening to the second lumen, the second openings to the first and second lumens being oriented to face in substantially opposite directions and wherein the second lumen comprises a closure for preventing blood leakage therealong.

Components of the invention may be included in medical kits providing the surgeon with the necessary equipment for performing the insertion of a VA-ECMO cannula sealed in a sterile environment.

Preferred embodiments of the invention will now be described, by way of example only, with reference to the accompanying drawings in which:

FIG. 1 shows a cross section of a first embodiment of the invention;

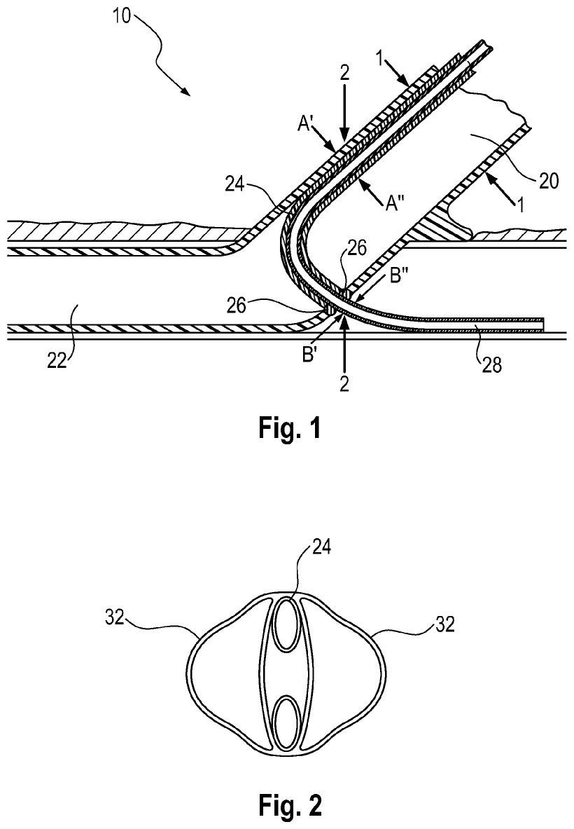

FIG. 2 is a transverse cross section of the FIG. 1 embodiment;

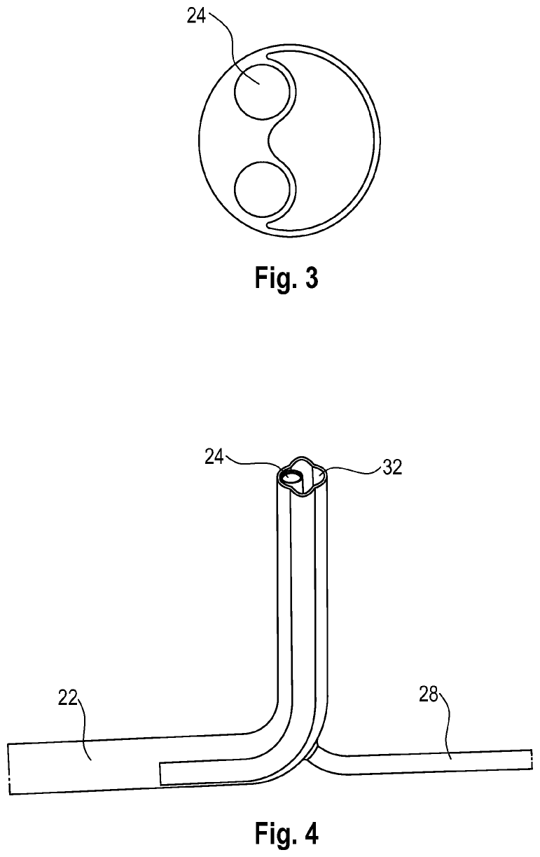

FIG. 3 is a transverse cross section of an alternative arrangement;

FIG. 4 is a schematic illustration of the first embodiment;

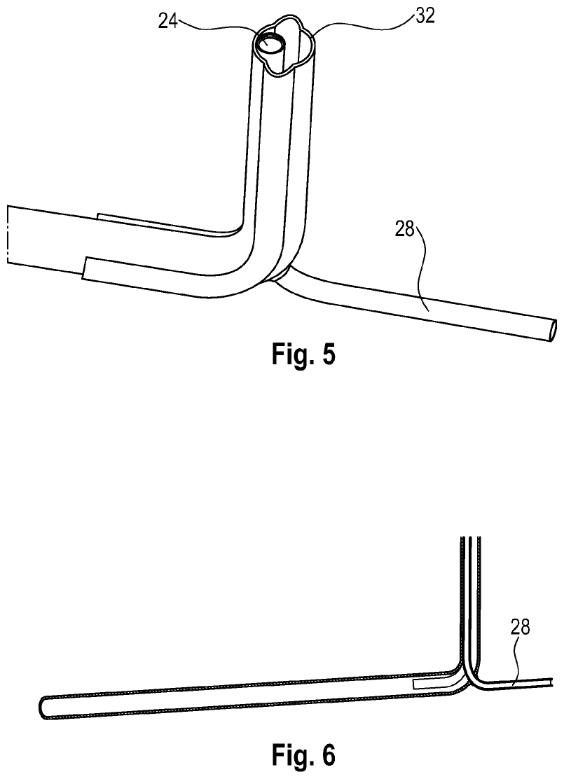

FIG. 5 is a detailed illustration of the first embodiment;

FIG. 6 is a cross sectional view of the first embodiment;

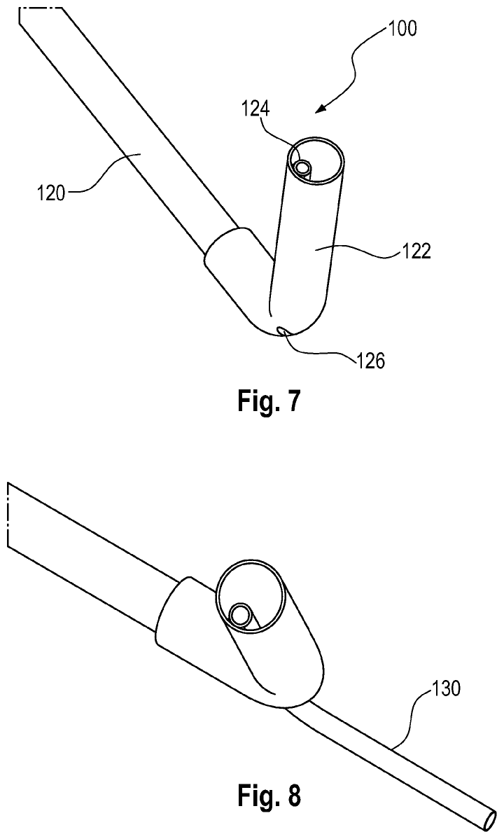

FIG. 7 is a schematic illustration of a third embodiment;

FIG. 8 is a further illustration of the third embodiment;

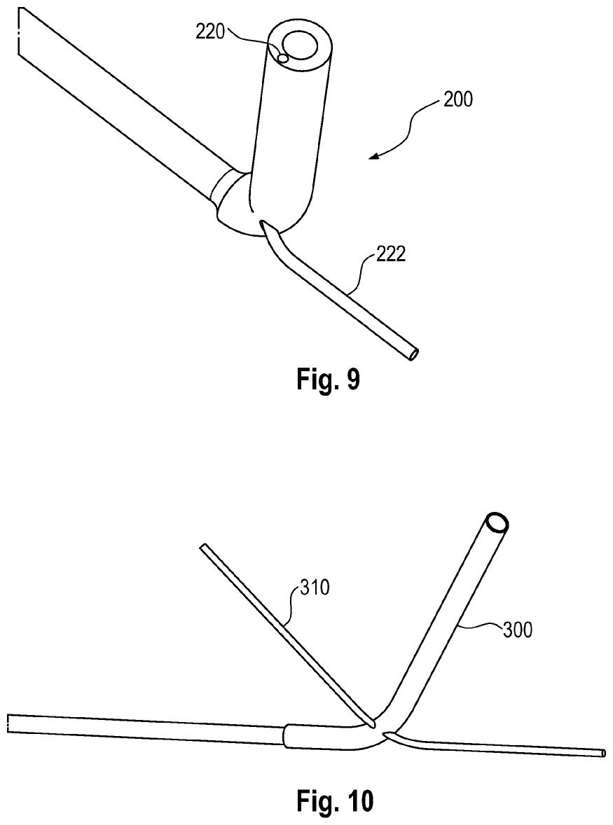

FIG. 9 is a schematic illustration of a fourth embodiment;

FIG. 10 is a schematic illustration of a fifth embodiment;

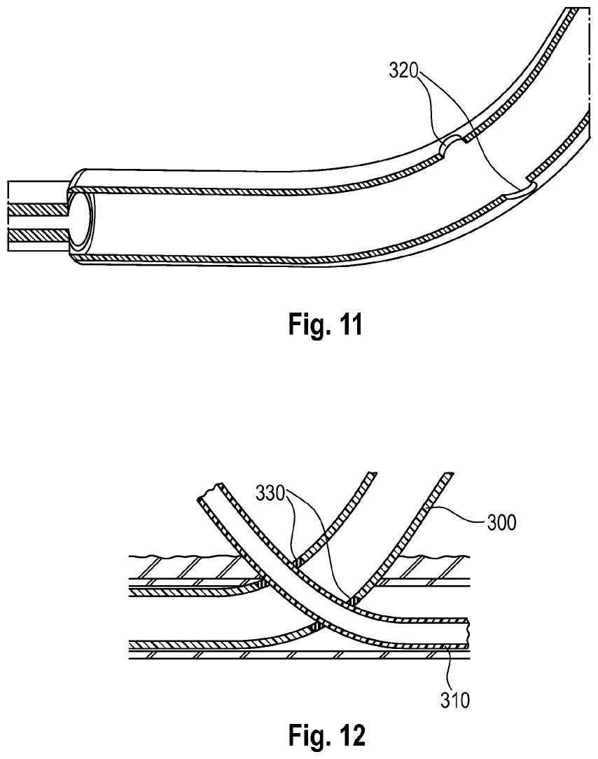

FIG. 11 is a detailed view of the fifth embodiment;

FIG. 12 is a cross sectional view of the fifth embodiment;

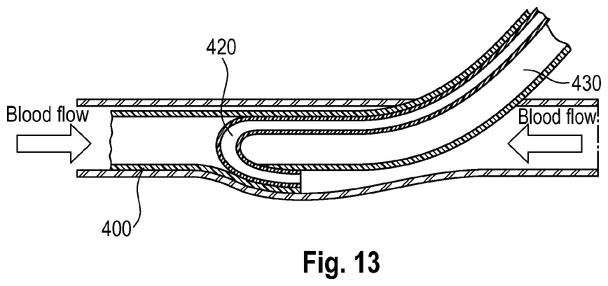

FIG. 13 shows a modification to the first embodiment of FIG. 5;

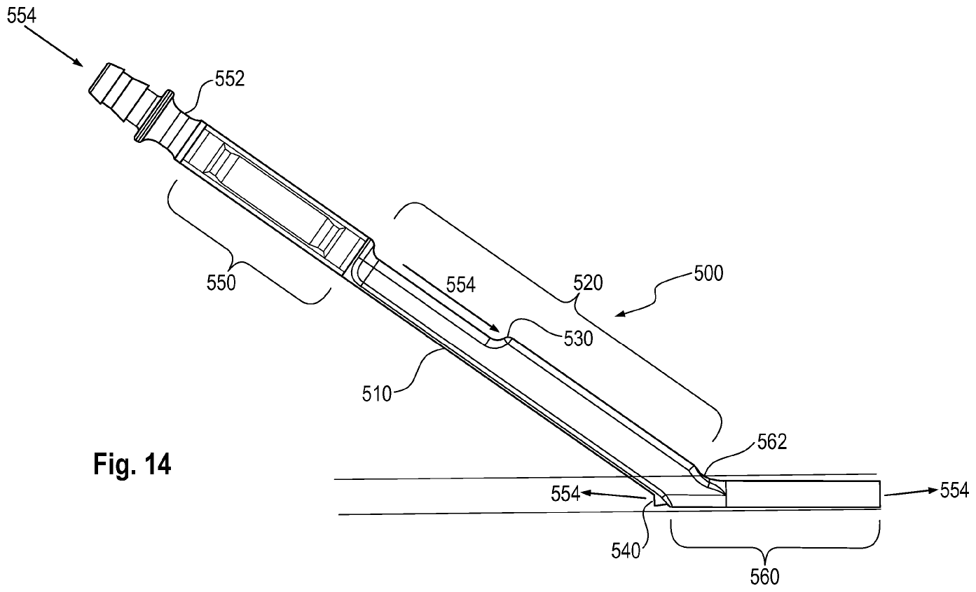

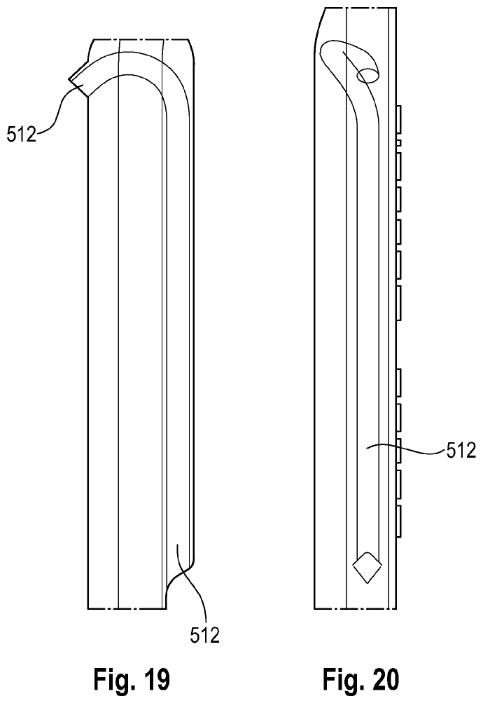

FIGS. 14-20 show a schematic illustration of a sixth embodiment;

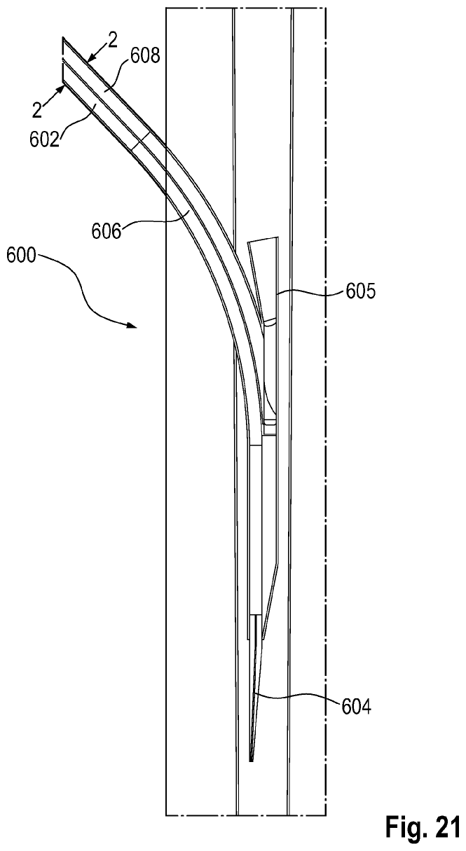

FIG. 21 shows a representation of a cannula according to a seventh embodiment of the invention inserted into an artery of a patient;

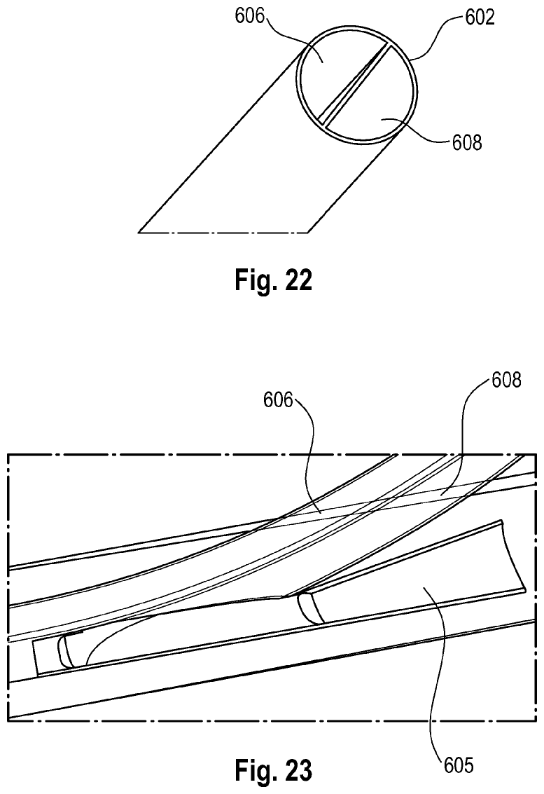

FIG. 22 shows a cross section of a main body of the cannula of FIG. 21;

FIG. 23 shows a detailed representation of a flexible tube of the cannula of FIG. 21;

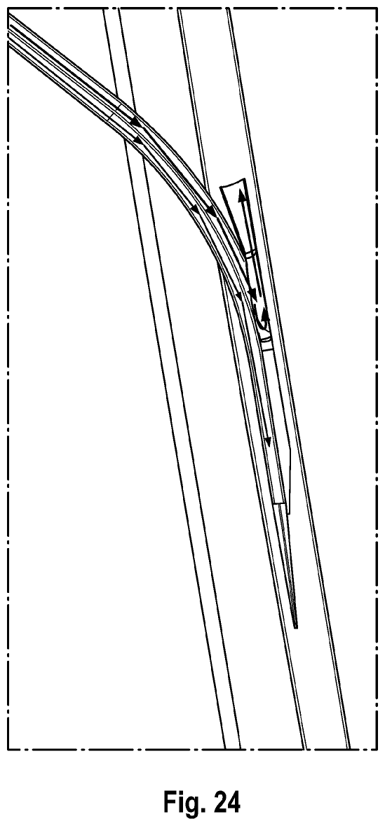

FIG. 24 illustrates a flow of blood through the cannula of FIG. 21;

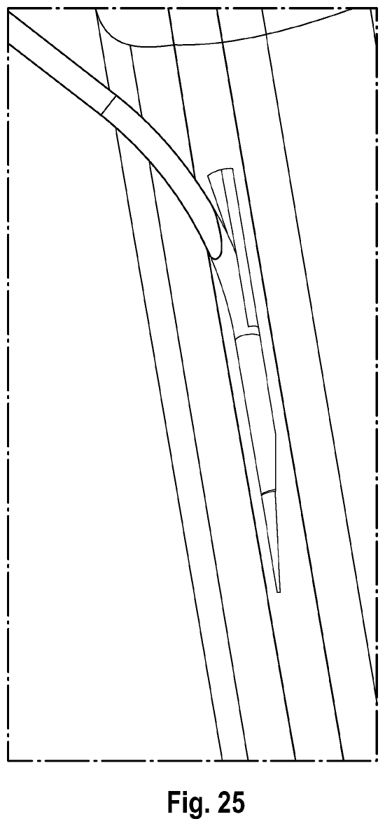

FIG. 25 is an isometric view of the cannula of FIG. 21 inserted into a patient

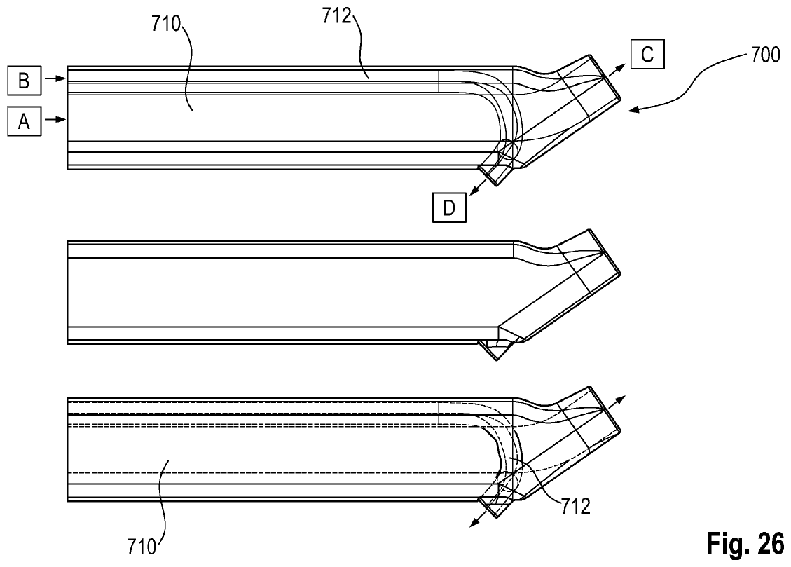

FIG. 26 shows a representation of a seventh embodiment of the invention in the form of a sleeve;

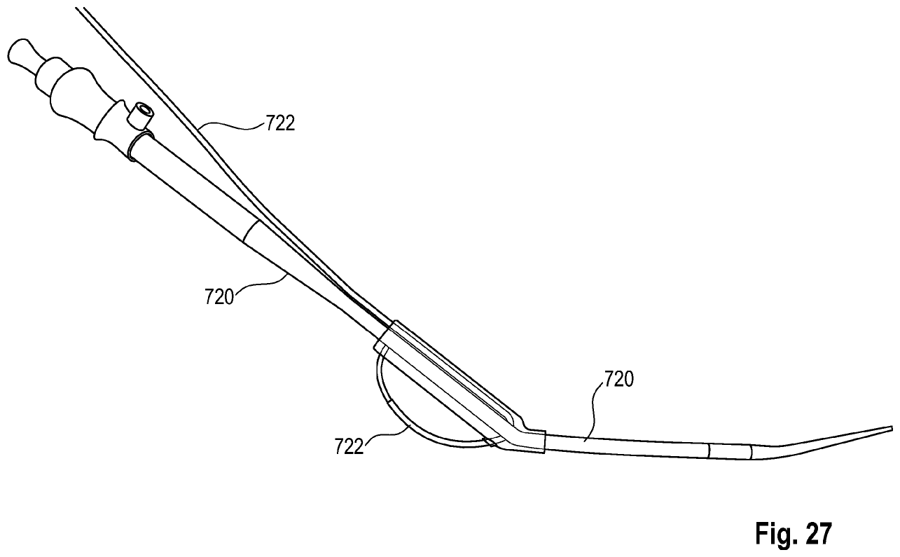

FIG. 27 is an image of the sleeve of FIG. 26 with inserted cannulas;

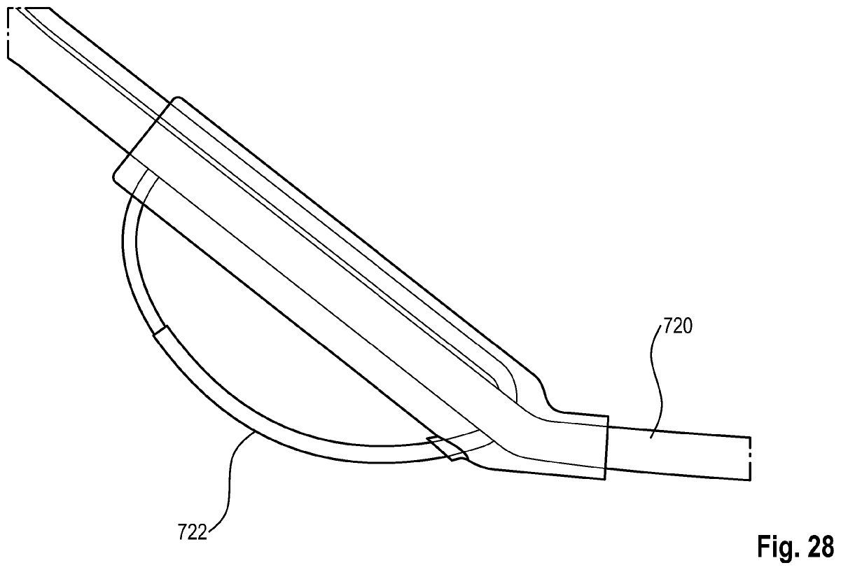

FIG. 28 is a detailed image of the arrangement of FIG. 27;

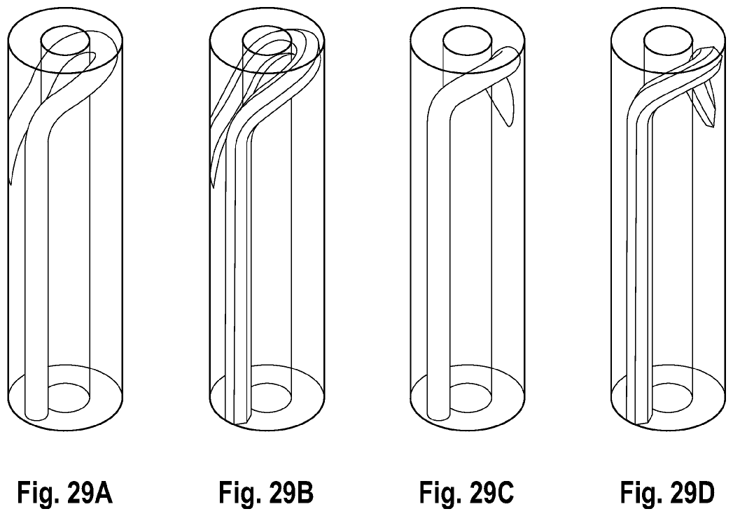

FIGS. 29A-29D show alternative lumen configurations for the sleeve of FIG. 26;

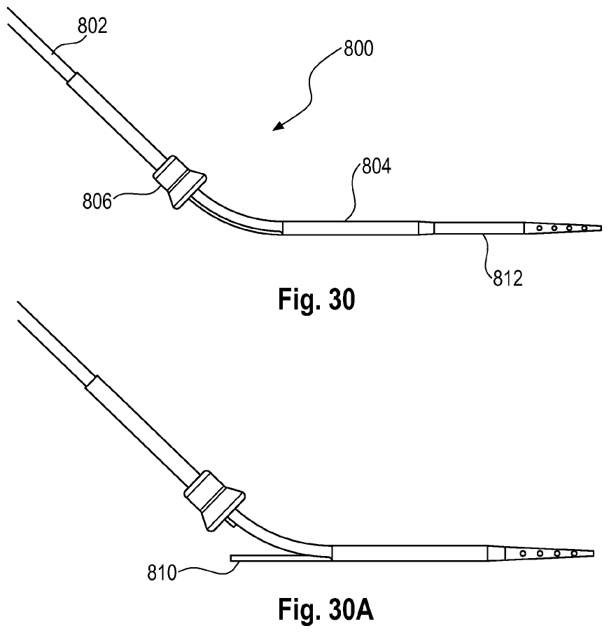

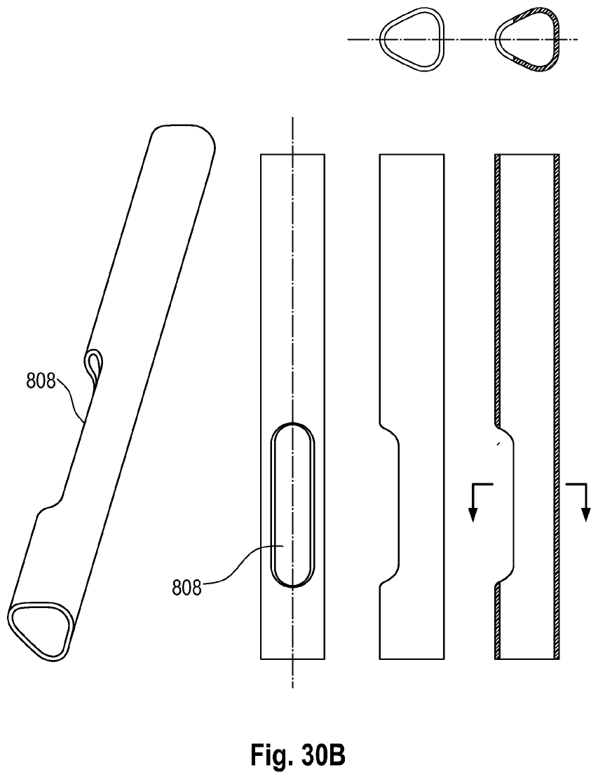

FIG. 30 shows a bi-directional perfusion system having a slidable sheath;

FIG. 30A shows the system of FIG. 30 with the sheath in a deployed position;

FIG. 30B shows varying views of the sheath of the system of FIG. 30;

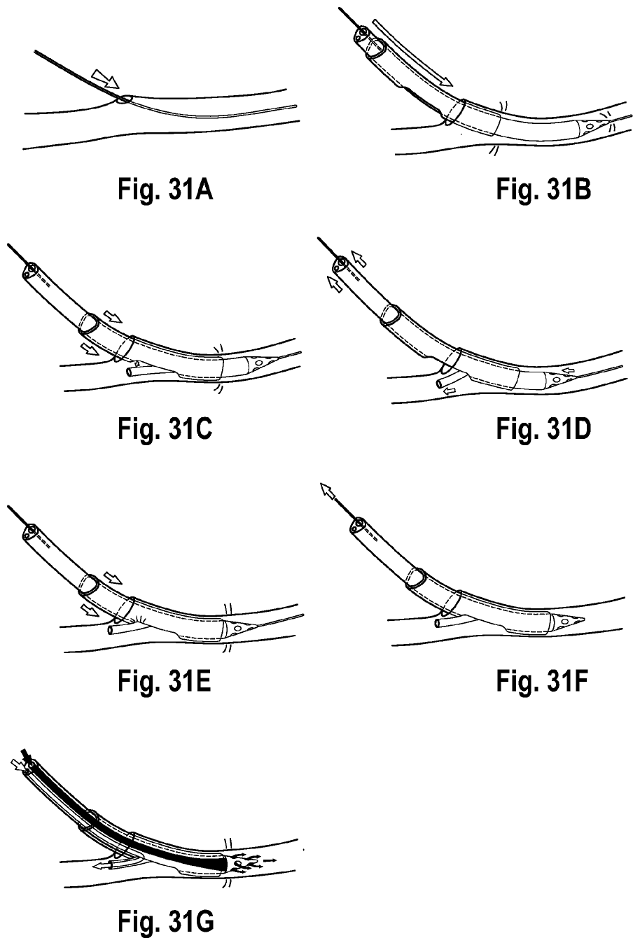

FIGS. 31A-31G show steps in the insertion of the system of FIG. 30 into a patient; and

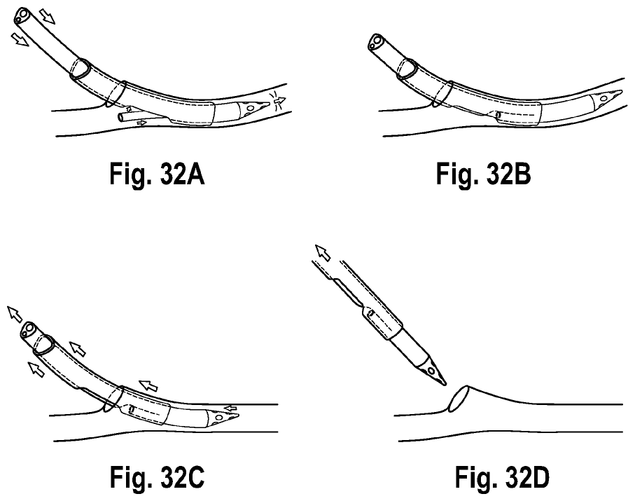

FIGS. 32A-32D show steps in the removal of the system of FIG. 30 from a patient.

Referring to FIG. 1, there is shown a section of a cannula 10 after insertion into a patient's vein for performing peripheral VA-ECMO. The cannula 10 has a main channel 20 leading to a primary arterial channel 22. The cannula 10 has a preformed channel 24 which terminates at a sealing ring 26. As shown, a distal reperfusion system cannula 28 (herein referred to as a secondary cannula) has been inserted into the preformed channel 24, passing through the sealing ring 26. A sealing ring (not shown) is provided at an entrance point of the distal reperfusion system cannula into the preformed channel 24.

As shown, in FIG. 1, the preformed channel 24 extends along the main channel along an upper part of a sidewall of the cannula 10. A cross section along the line 1-1 would show the preformed channel at the 12 o'clock position and a projected position of an exit of the preformed channel from the cannula 10 would be at the 6 o'clock position, and accordingly angularly offset from an entrance of the preformed channel.

As shown in FIG. 1, the secondary cannula 28 is inserted into the preformed channel 24 to provide anterograde perfusion.

As the primary arterial channel 22 is for providing a flow of blood in an opposite direction to the normal flow of blood, a relatively high pressure would be required to achieve sufficient blood flow compared with a pressure to achieve blood flow in the anterograde direction. Accordingly, it is beneficial to provide two cannulas with separate supplies of blood since the pressure to achieve sufficient flow along the primary arterial channel 22 will be higher than that along the secondary cannula.

As shown, the primary arterial cannula may have a diameter of 19-21 Fr while the body outside the patient comprising the main channel and the preformed channel may have a diameter of 25 Fr. It is envisaged that the reperfusion system cannula 28 may have a diameter of 6-8 Fr.

A particular advantage of such an arrangement is that there is no limitation on the secondary, or distal reperfusion, cannula's outside diameter, since the secondary cannula is crossing the primary arterial cannula towards the anterograde direction. Additionally, the increased diameter of the outside-the-body main arterial cannula portion allows flexibility in the secondary cannula size selection, without having to increase the total outer diameter of the main arterial cannula. Therefore, the secondary cannula outer diameter is not added to the primary arterial cannula outer diameter and thus the risk of damaging the arterial wall is reduced.

FIG. 2 shows a cross section of the cannula 10 along a line 2-2 of FIG. 1. As shown, the outer portion has a non-round cross section, with a pair of “wings” 32 to provide increased cross-sectional area to compensate for the area of the preformed channel 24. As shown, the preformed channel runs along the outer body on an opposite side to the exit port formed by the sealing ring 26. While the preformed channel 24 itself has a circular cross section, because the line 2-2 cuts the preformed channel 24 at an angle, in FIG. 2 the cross-section of the preformed channel 24 has an oval shape.

An alternative arrangement, as shown in FIG. 3 has the preformed channel 24 on the same side as the exit port. Compared with the arrangement of FIG. 2, the arrangement of FIG. 3 has an advantage that a dilator can pass through the main channel 20 unhindered by the preformed channel 24. In the case of the FIG. 2 arrangement, either a reduced cross sectional dilator would be required or one having a slit shape allowing it to pass on both sides of the preformed channel 24.

Further representations of the cannula 10 are shown in FIGS. 4-6.

A further configuration is shown in FIG. 7. In the arrangement of FIG. 7, a cannula 100 has a primary arterial cannula in-the-body portion 120 of diameter 21 Fr for insertion in the retrograde direction. An out-of-body portion 122 of the cannula 100 has an increased diameter of 29 Fr and within the portion 122 is arranged a preformed channel 124 of diameter sufficient to allow the passage of a distal arterial cannula therethrough, the preformed channel 124 terminating at a channel exit 126 which incorporates a seal (not shown). FIG. 8 shows the same arrangement with the distal arterial cannula 130 inserted. The preformed channel extends along the cannula 100 at the 9 o'clock position and exits the cannula at an angular position between 9 o'clock and 6 o'clock,

A still further alternative configuration of a cannula 200 is shown in FIG. 9 in which a preformed channel 220 is formed in a thickened wall of an out-of-body portion of the cannula. A 6 Fr distal arterial cannula 222 passes through the preformed channel 220 and emerges in the anterograde direction.

The arrangement of FIG. 9 has an advantage that blood flowing through the main central channel does not become turbulent as it is not disturbed by the preformed channel 220.

Compared with the arrangement of FIG. 3, the wall of the main channel has a smooth, regular internal surface, helping to prevent turbulence in the flow of blood.

FIGS. 10, 11 and 12 show another arrangement of a cannula 300. A secondary cannula 310 crosses the primary arterial cannula 300 towards the retrograde direction. The primary arterial cannula includes two through holes 320, each closed by a sealing ring 330. After the primary arterial cannula is positioned in the patient, the distal arterial cannula 310 is inserted in the anterograde direction through the sealing rings 330. An in-body portion of the cannula 300 may have a diameter of 21 Fr, an out-of-body portion 29 Fr and the distal arterial cannula 310 may have a diameter of 8 Fr. As may be seen from FIG. 11, there is no preformed channel present but this may be modified to include such a channel.

The secondary cannula may either cross the main channel centrally or off-center. Alternatively, the secondary cannula may pass through a channel in the external wall of the main channel. If the secondary cannula crosses the main channel, blood flow turbulence may result. Unlike with the preformed channel, an arrangement in which a secondary cannula crosses the main channel does not adversely affect the design of the dilator since the dilator is removed prior to the secondary cannula being inserted.

FIG. 13 shows a variation of the arrangement of FIGS. 5-8. In the arrangement of FIG. 13, a preformed channel 420 extends beyond a sidewall 430 of a main arterial cannula 400. A secondary cannula (not shown) is advanced along the preformed channel 420 using a scrolling system (not shown).

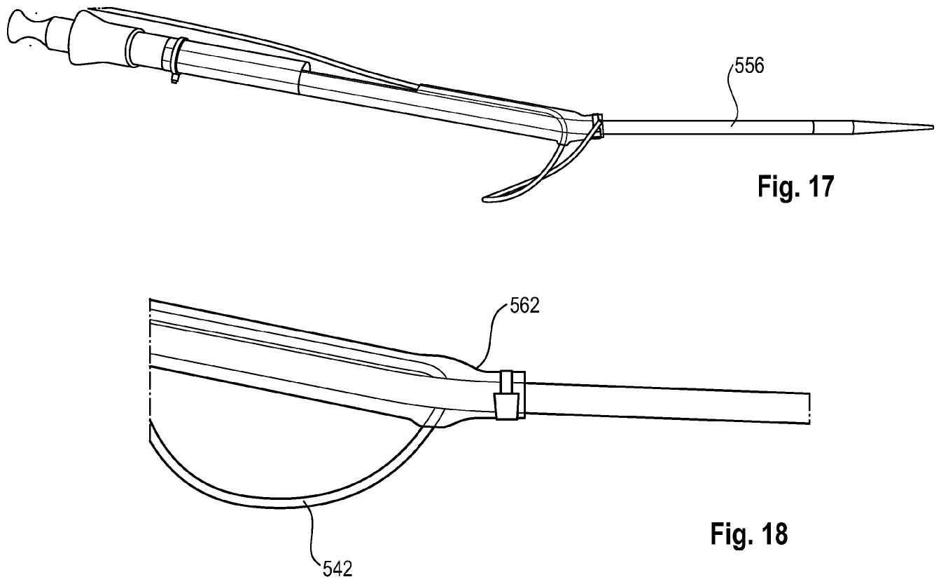

A yet further embodiment is illustrated in FIGS. 14-20. A bi-directional perfusion system 500 comprises a main cannula 510 having a lumen therethrough for the supply of blood in a retrograde direction in the patient's artery. The main cannula 510 includes a preformed channel 512 in a rigid part of a sidewall of the main cannula along a portion 520 with an entrance indicated by arrow 530 and an exit indicated by arrow 540. The preformed channel is configured to allow a secondary or reperfusion cannula 542 to pass through it for providing an anterograde blood supply. The main cannula 510 includes a flexible or pliable region 550 which may be clamped after the cannula has been de-aired. A silicone valve 552 is also provided for sealing around an insertion dilator 556 shown in FIG. 17. In FIG. 14, arrows 554 show the direction of blood flow in the system 500.

A intra-arterial portion 560 of the main cannula 510 is positioned within the patient's artery. An angle of approximately 35° may be present between the inter-arterial portion 560 and the rest of the cannula which is external to the patient.

The portion 520 of the cannula 510 is fabricated from relatively stiff and inflexible material in order to aid the passage of the secondary cannula 542 through the preformed channel 512. The intra-arterial portion 560 may be formed from a soft, flexible material, such as silicone rubber, and may be reinforced with metal tubing at a distal portion to provide stiffness to aid insertion into the patient's artery. A flexible region 562 provides a hinge between the portion 560 and the portion 520.

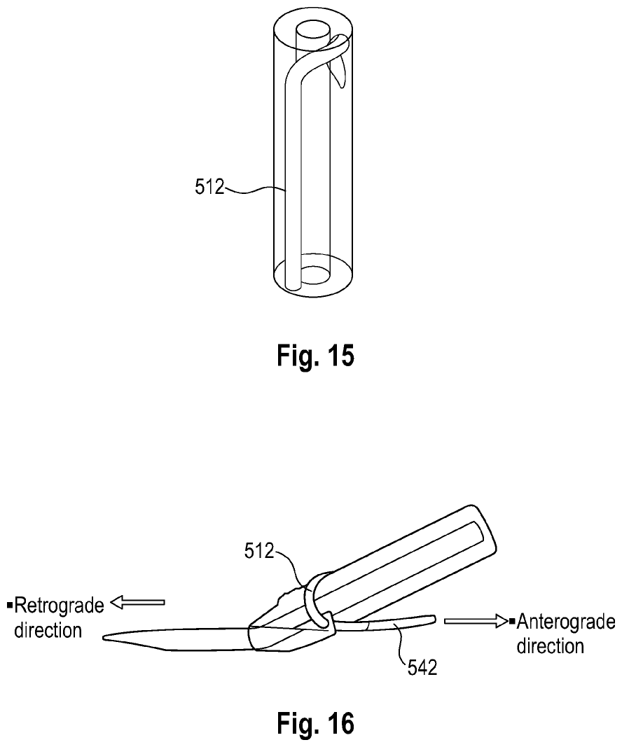

Referring to FIG. 15, the course of the preformed channel 512 through the sidewall of the cannula 510 is shown. the preformed channel extends along the cannula 510 in a first part and then extends around the wall of the cannula 510 before exiting in the anterograde direction. This trajectory increases the radius of curvature to aid in the passage of the secondary cannula therethrough.

FIG. 21 shows a bi-directional perfusion ECMO cannula 600 according to another embodiment of the present invention inserted into a femoral artery of a patient. The cannula 600 comprises a main body 602. As shown in FIG. 21 an over the wire dilator 604 is used to insert the cannula into the femoral artery. Attached to the main body 602 is a flexible tube 605 extending in the anterograde direction.

FIG. 22 shows a cross section of the main body 602 along the line 2-2 of FIG. 21. The main body has a superior lumen 606 for providing retrograde blood perfusion and an inferior lumen 608 for achieving anterograde blood perfusion. The inferior lumen 608 leads to the flexible tube 605. The superior lumen 606 and the inferior lumen 608 have a cross-sectional area ratio which may be 70:30 but other ratios are possible, for example 80:20 or 90:10. In general a blood flow of 5000 cm³/min might be delivered via the superior lumen and a blood flow of 200 cm³/min might be delivered via the inferior lumen.

Referring again to FIG. 21, the dilator 604 tapers to a point and preferably has a half-moon or semi-circular cross section for removal via the superior lumen. The flexible tube 605 has a soft, flexible thin-walled construction and may be fabricated of the same materials used for balloon catheter construction such as polyurethane or silicone rubber.

For insertion into the patient, the flexible tube 605 is partially wrapped around the main body 602 which preferably has a braided or coiled wall. Once correctly inserted into the patient, blood or other fluid is introduced into the inferior lumen 608 causing the flexible tube to expand and assume the position shown in FIG. 23. Preferably, an exit port of the flexible tube 605 is initially sealed, with this seal being opened by the introduction of the blood or other fluid. It is preferable that a marker is mounted at the free end of the flexible tube 605 for enabling the position of this end to be determined in the patient before the tube is expanded by the introduction of blood or other fluid. Such a marker may be a radiopaque marker which may be identified using X-ray imaging techniques. A suitable blood detector could also be used for determining the correct positioning of the flexible tube 605 prior to expansion.

Once the cannula 600 of FIG. 21 is inserted, the flow of blood into the patient is shown in FIG. 24.

It is preferred that the cannula 600 be supplied as a kit including a cannula for the insertion of a guide wire, the guide wire itself and the cannula of the invention including the dilator 604.

A still further alternative embodiment of the present invention is shown in FIG. 26, showing a bi-directional perfusion sleeve 700 which is a development of the system 500 of FIGS. 14-20. The bi-directional perfusion sleeve 700 in combination with a retrograde perfusion catheter and an anterograde reperfusion catheter provides a bi-directional perfusion system. The perfusion sleeve 700 has a central lumen 710 and a peripheral lumen 712. The peripheral lumen 712 extends parallel to the central lumen 710 for a portion of a length of the perfusion sleeve 700. In use the perfusion sleeve 700 is placed over a retrograde perfusion cannula (not shown) such that the retrograde perfusion cannula providing retrograde blood perfusion extends through the central lumen 710 from A to C in FIG. 26. A separate anterograde reperfusion cannula (not shown) for providing anterograde perfusion is inserted along the peripheral lumen 712 from B to D.

The bi-directional perfusion sleeve 700 is essentially a sleeve which is placed over the retrograde perfusion catheter which may be a “Nextgen Bio Medicus” cannula of size 19 Fr. The central lumen 710 is dimensioned to accept the retrograde perfusion catheter and is of matching size. The use of a suitable lubrication medium or saline fluid eases the positioning process. Further movement of the catheter within the lumen is restricted by friction or the provision of barbs.

Once the bi-directional perfusion sleeve 700 has been positioned over the retrograde perfusion catheter, the assembly is inserted into the artery of the patient, for example using the Seldinger technique over a previously inserted guide wire. Thereafter, the anterograde reperfusion cannula is inserted through the peripheral lumen 712 such that it exits the perfusion sleeve 700 and extends in the anterograde direction along the patient's artery, providing anchoring of the cannula and enabling anterograde perfusion. FIGS. 27 and 28 show images of the perfusion sleeve 700 after both the retrograde and anterograde perfusion cannulas have been inserted.

Referring to FIG. 27, shows the bi-directional perfusion sleeve 700 with both a retrograde perfusion cannula 720 and an anterograde reperfusion catheter 722 inserted into and exiting from the respective lumens of the sleeve 700. The retrograde perfusion cannula 720 extends in the retrograde direction and the anterograde reperfusion catheter 722 extends in the anterograde direction.

Advancement of the reperfusion catheter through the peripheral lumen 712 is aided by a low coefficient of friction. Parts of the bi-directional perfusion sleeve 700 which are inserted into the patient should be without sharp edges and formed of a silicone resin. Soft materials are preferred so as to ensure the sleeve is atraumatic to the patient. A transition from soft to hard materials may be provided which may provide a degree of hinging between an intra-arterial portion and an external portion.

The peripheral lumen 712 may have one of a number of configurations. FIGS. 29A-29D show different arrangements of the peripheral lumen within the bi-directional perfusion sleeve 700. FIG. 29A shows an arrangement in which the peripheral lumen has a circular cross section and loops around the central lumen by 335°. FIG. 29B shows the same configuration but with a peripheral lumen having a hexagonal cross section. Similarly, FIGS. 29C and 29D show circular hand hexagonal cross section peripheral lumens which curve around the central lumen by a lesser degree, for example 180° as shown. The degree of wrap-around may be between 150° and 360°, preferably between 180° and 335°.

The configuration of the peripheral lumen around the central lumen allows for a more fluent, less aggressive trajectory which eases the advancement of the reperfusion cannula due to reduced friction and decreases the risk for blood activation. Furthermore, the geometry of the reperfusion channel also influences the friction between reperfusion channel and catheter. The hexagonal shape cross section will reduce the contact surfaces with the catheter, so the friction is reduced. Finally, the trajectory assures a correct aligned opening and herewith advancement with the centerline of the femoral artery herewith preventing that the reperfusion cannula might scrape or damage the arterial wall.

The peripheral lumen has a closure such as a stop, a seal or a one-way valve which prevents leakage of the blood both before the insertion of the reperfusion catheter and after insertion along the reperfusion catheter. Since there is a prevention to avoid blood leakage it is also possible to use the bi-directional cannula as a standard cannula, using a catheter inserted through the central channel only.

A further embodiment of a bi-directional perfusion system 800 is shown in FIG. 30. The system 800 comprises a bi-directional cannula 802 and a sheath 804, the cannula 802 being slidable within the sheath 804. The bi-directional cannula may be similar to the cannula 600 shown in FIG. 21. A slidable stop or bumper 806 is also provided for skin contact and to fix an inserted position. End stops (not shown) are provided to limit the movement of the sheath 804 over the cannula 802. FIG. 30 shows the system 800 with the sheath in a position whereby an anterograde cannula leg 810 is secured and FIG. 30A shows the system with the sheath in a position whereby the anterograde cannula leg 810 is released.

The sheath 804 incorporates a cut out region 808. When the cannula 802 is withdrawn along the sheath 804 (or the sheath is advanced), the anterograde cannula leg 810 is released from a main cannula 812 providing retrograde perfusion and adopts a position suitable or enabling anterograde reperfusion of the patient. The main cannula 812 may be withdrawn slightly to ensure optimal positioning of the anterograde cannula leg 810 within the artery of the patient. Preferably, the anterograde cannula leg 810 exhibits a memory-effect causing it to deflect away from the main cannula 812 once the anterograde cannula leg 810 is exposed by the cut out region 808. The sheath 804 is shown in more detail in FIG. 30B. As indicated, the sheath may have an ovoid cross section corresponding to a cross-sectional shape of the bi-directional cannula 802 but both may also have a circular cross section. Preferably, the sheath is relatively rigid, fabricated for example from PTFE. The cannula 802 may have two lumens but this is not necessary and the cannula may have a single lumen providing blood supply in both anterograde and retrograde directions.

Withdrawal of the bi-directional perfusion system 800 from the patient is achieved by withdrawing the sheath 804 which causes the anterograde cannula leg 810 to be re-secured within the sheath 804 after which the system 800 is withdrawn from the patient.

Various steps showing the insertion of the bi-directional perfusion system 800 into a patient are shown in FIGS. 31A-31G. In FIG. 31A a guidewire is inserted in a retrograde direction into the patient. In FIG. 31B, the system 800 with the cannula 802 and the sheath 804 are inserted over the guidewire into the retrograde vessel. In FIG. 31C the sheath is held in position and the cannula is pulled back to release anterograde cannula leg. In FIG. 31D, the sheath is held in position and cannula pulled back to slide and insert the deflected anterograde cannula leg into the anterograde vessel. FIG. 31E shows an optional stage in which the the sheath is moved forward to lock the deflected anterograde cannula leg in position. In FIG. 31F, the guide wire is withdrawn. FIG. 31G shows the system 800 providing retrograde and anterograde blood flow.

Various steps showing the removal of the bi-directional perfusion system 800 from a patient are shown in FIGS. 32A-32D. In FIG. 32A the cannula 802 is slid forward in the sheath 804 to cover and retract the anterograde cannula leg 810 inside the sheath. In FIG. 32B the anterograde cannula leg 810 is secured within the sheath 804 ready for retraction. In FIG. 32C the system 800 is withdrawn from the artery until it is fully withdrawn as shown by FIG. 32D.

1. A bi-directional perfusion cannula for use in peripheral veno-arterial extracorporeal membrane oxygenation of a patient, the cannula comprising a cannula main body having a primary lumen, the primary lumen leading to a distal end of the cannula main body for providing retrograde blood perfusion, characterized in that the main body further comprises a passage for passing a second cannula therethrough, the passage being oriented such that when a second cannula is inserted into the passage the second cannula is arranged for providing anterograde blood perfusion and wherein the passage enters the main body at a first position on a side wall of the main body and leaves the main body at a second position on the side wall of the main body, the first position and the second position being angularly offset from one another about a projected cross section of the main body.

2. The bi-directional perfusion cannula according to claim 1, wherein the passage is a preformed channel having a sidewall which leads from the first position to the second position.

3. The bi-directional perfusion cannula according to claim 2, wherein the preformed channel is attached to the sidewall of the main body along a portion thereof.

4. The bi-directional perfusion cannula according to claim 3, wherein the preformed channel is attached to an upper portion of the sidewall of the main body and the second position is located at a lower portion of the sidewall of the main body.

5. The bi-directional perfusion cannula according to claim 3, wherein the preformed channel is attached to a side portion of the sidewall of the main body and the second position is located at a lower portion of the sidewall of the main body.

6. The bi-directional perfusion cannula according to claim 1, wherein the passage is terminated by a seal.

7. The bi-directional perfusion cannula according to claim 1, wherein a distal portion of the main body has a generally circular cross section and a second portion of the main body including the passage has a non-circular cross section with at least one protrusion providing an increased cross sectional area compared with the distal portion.

8. The bi-directional perfusion cannula according to claim 1, wherein a distal portion of the main body has a generally circular cross section and a second portion of the main body including the passage has a generally circular cross section with the second portion having an increased cross sectional area compared with the distal portion.

9. The bi-directional perfusion cannula according to claim 1, wherein the passage is within a sidewall of the main body.

10. The bi-directional perfusion cannula according to claim 1, wherein the main body has through holes on either side of the main body, the through holes each including a seal and providing the passage for the second cannula therethrough.

11. (canceled)

12. (canceled)

13. A bi-directional perfusion cannula for use in peripheral veno-arterial extracorporeal membrane oxygenation of a patient, the cannula comprising a cannula main body having a superior lumen and an inferior lumen, wherein the superior lumen extends to a distal end of the cannula for providing retrograde blood perfusion, wherein
the cannula further comprises a flexible tube extending from the cannula main body in a direction away from the distal end, the flexible tube being in fluid communication with the inferior lumen for providing anterograde blood perfusion.

14. The cannula according to claim 13, wherein the superior lumen has a greater cross-sectional area than the inferior lumen.

15. (canceled)

16. The cannula according to claim 13, wherein the flexible tube has a wall thickness which is less than a wall thickness of the cannula main body.

17. The cannula according to claim 13, wherein the flexible tube includes position indicating means placed vicinal to a proximal outlet of the tube for enabling an operator to determine a position of the flexible tube proximal outlet within the patient.

18. The cannula according to claim 17, wherein the position indicating means is one of a marker identifiable by ultrasound or x-ray scanning, or a blood detector.

19. (canceled)

20. (canceled)

21. The cannula according to claim 13, wherein in a non-inflated state the flexible tube is wrapped around an outer surface of the main body.

22. The cannula according to claim 13, further comprising a retractable dilator positioned at the distal end of the cannula.

23. (canceled)

24. The cannula according to claim 13, further comprising a sheath around and slidable over the cannula main body, the sheath including a cut out portion dimensioned such that the flexible tube can extend away from the cannula main body when the sheath is in a position such that the cut out portion is aligned with the flexible tube.

25. (canceled)

26. (canceled)

27. A bi-directional perfusion sleeve having a first lumen for receiving a retrograde perfusion catheter to form a reperfusion cannula and a second lumen for receiving an anterograde perfusion catheter to form a bi-directional cannula, the sleeve having at a first end a first opening to the first lumen and a first opening to the second lumen and at a second end a second opening to the first lumen and between the first end and the second end a second opening to the second lumen, the second openings to the first and second lumens being oriented to face in substantially opposite directions and wherein the second lumen comprises a closure for preventing blood leakage therealong.

28. The bi-directional perfusion sleeve according to claim 27, wherein the first lumen is a central lumen and the second lumen is a peripheral lumen.

29.-36. (canceled)
